(19) United States
(12) Patent Application Publication
(10) Pub. No.: US 2025/0180554 A1
(43) Pub. Date: Jun. 5, 2025

(54) RESIN PARTICLE AND AFFINITY PARTICLE INCLUDING THE SAME

(71) Applicant: CANON KABUSHIKI KAISHA, Tokyo (JP)

(72) Inventors: TARO ENDO, Kanagawa (JP); NORISHIGE KAKEGAWA, Tokyo (JP); KENJI KITAGAWA, Tokyo (JP); TEIGO SAKAKIBARA, Tokyo (JP)

(21) Appl. No.: 18/968,457

(22) Filed: Dec. 4, 2024

(30) Foreign Application Priority Data
Dec. 5, 2023 (JP) 2023-205719
Nov. 19, 2024 (JP) 2024-201551

(51) Int. Cl. G01N 33/545; C08K 5/00; C08L 25/08; C09K 11/02; C09K 11/06

(52) U.S. Cl. CPC G01N 33/545; C08K 5/0091; C08L 25/08; C09K 11/02; C09K 11/06; C08L 2207/53; C08L 2312/00; C09K 2211/1007; C09K 2211/1014; C09K 2211/1018; C09K 2211/182

(57) ABSTRACT

A resin particle having a core-shell structure includes a europium complex, a core portion, and a shell portion, wherein the core portion contains a specific copolymer, the shell portion contains a specific polymer, and at least one of the core portion and the shell portion includes a cross-linked structure.

### BACKGROUND

### Technical Field

The present disclosure relates to a resin particle and an affinity particle including the same.

### Description of the Related Art

In recent years, in fields of medicine and clinical examinations, a very small amount of living-body component in, for example, blood or a sampled portion of a organ has become able to be detected with high sensitivity. Japanese Patent Laid-Open No. 2005-171097 discloses a resin fine particle, wherein a molecular recognition body, such as an antigen or an antibody, is fixed to the particle and used in fields of analytical reagents or diagnostic drugs.

### SUMMARY

The present disclosure provides a resin particle that does not readily cause nonspecific adsorption when being used to detect a target substance such as an antigen. In particular, the present disclosure provides a resin particle having high sensitivity when a target substance is detected by using a fluorescent depolarization method.

A resin particle having a core-shell structure according to the present disclosure includes a europium complex, a core portion, and a shell portion, wherein the core portion contains a copolymer having a unit denoted by Formula (CORE_1), the shell portion contains a polymer having a unit denoted by Formula (SHELL_1) and a polymer having a unit denoted by Formula (SHELL_2), and at least one of the core portion and the shell portion includes a cross-linked structure.

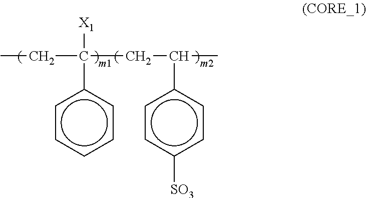

In Formula (CORE_1), X₁ represents H or CH₃, each of m1 and m2 represents an integer of 1 or more, and m1 and m2 satisfy 10<m1/m2.

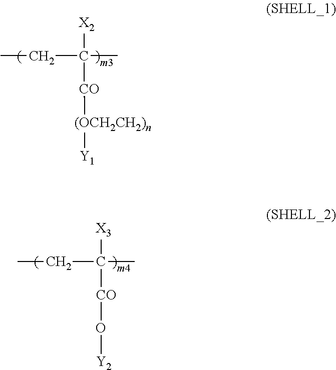

In Formula (SHELL_1) and Formula (SHELL_2), each of X₂ and X₃ represents H or CH₃, Y₁ represents OH or OCH₃, Y₂ represents Formula (SHELL_3) or CH₂CH₂OH, each of m3 and m4 represents an integer of 1 or more, and n represents an integer of 1 or more and 40 or less.

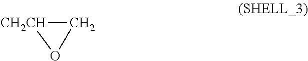

Further features of the present disclosure will become apparent from the following description of exemplary embodiments with reference to the attached drawings.

### BRIEF DESCRIPTION OF THE DRAWINGS

FIG. 1 is a schematic diagram illustrating the structure of a resin particle according to an embodiment of the present disclosure.

FIG. 2 is a schematic diagram illustrating the structure of a resin particle in the related art.

### DESCRIPTION OF THE EMBODIMENTS

The present disclosure will be described below in detail with reference to embodiments, but the invention is not limited to these embodiments.

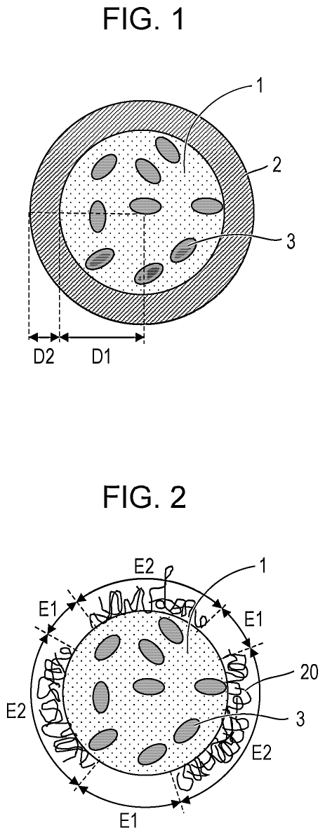

A resin particle according to the present embodiment will be described in detail with reference to FIG. 1.

Disadvantages addressed by resin particle according to the present embodiment

As a result of the investigation by the present inventors, it was found that when a resin particle disclosed in Japanese Patent Laid-Open No. 2005-171097 is used, a nonspecific adsorption disadvantage may occur. The nonspecific adsorption disadvantage is a disadvantage that, regarding the resin particle and an inspection target component, unintentional adsorption between the particle and an impurity rather than specific adsorption between the particle and a substance to be detected (target substance such as an antigen) occurs. That is, the present embodiment provides a resin particle that does not readily cause nonspecific adsorption when being used to detect a target substance such as an antigen. In particular, the present embodiment provides a resin particle having high sensitivity when a target substance is detected by using a fluorescent depolarization method.

As illustrated in FIG. 1, the resin particle according to the present embodiment includes a europium complex 3 and has a core-shell structure. The core-shell structure is configured to include a core portion 1 and a shell portion 2 covering the surface of the core portion 1. It can also be said that the resin particle according to the present embodiment is a resin particle including a first layer and a second layer in this order. The first layer may be a particle, and it can also be said that the first layer of the resin particle is the core portion, and the shell portion is the second layer. In this regard, in the present embodiment, a layer different from the second layer may be included between the first layer and the second layer and further outside the second layer (resin particle surface side).

Hereafter, regarding the resin particle according to the present embodiment, a resin particle including a first layer and a second layer in this order will be described with reference to a configuration in which the first layer is a core portion, and the second layer is a shell portion, as an example.

In FIG. 1, D1 represents the radius of the core portion, and D2 represents the thickness of the shell portion. In the present embodiment, the core portion contains a copolymer denoted by Formula (CORE_1) below. Herein, the copolymer is typically a random copolymer but may be a block copolymer within the bounds of obtaining the effect of the present disclosure.

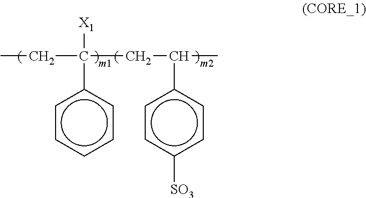

In Formula (CORE_1), X₁ represents H or CH₃, each of m1 and m2 represents an integer of 1 or more, and m1 and m2 satisfy 10<m1/m2.

In the present embodiment, the core portion can contain a copolymer denoted by Formula (CORE_1) as a main component.

In the present embodiment, the shell portion contains a polymer having a unit denoted by Formula (SHELL_1) below and a polymer having a unit denoted by Formula (SHELL_2) below.

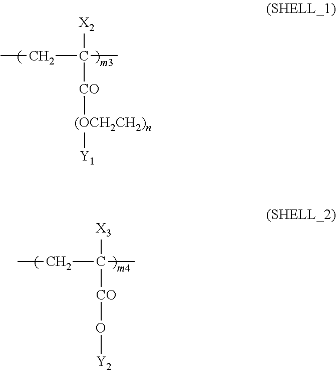

In Formula (SHELL_1) and Formula (SHELL_2), each of X₂ and X₃ represents H or CH₃, Y₁ represents OH or OCH₃, Y₂ represents Formula (SHELL_3) below or CH₂CH₂OH, each of m3 and m4 represents an integer of 1 or more, and n represents an integer of 1 or more and 40 or less.

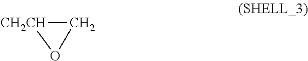

In this regard, the shell portion can contain a polymer having a unit denoted by Formula (SHELL_1) and a polymer having a unit denoted by Formula (SHELL_2) as main components.

In addition, in the present embodiment, at least one of the core portion and the shell portion includes a cross-linked structure. In this regard, in the present embodiment, both the core portion and the shell portion may include a cross-linked structure.

Further, the polydispersity index of the resin particle according to the present embodiment may be 0.1.

The resin particle according to the present embodiment has such a configuration and does not readily cause nonspecific adsorption when being used to detect a target substance such as an antigen. In particular, the resin particle has high sensitivity when a target substance is detected by using a fluorescent depolarization method.

The shell portion according to the present embodiment contains, for example, at least one selected from the group consisting of a polymer of hydroxyethyl methacrylate, a polymer of polyethylene glycol monomethyl ether methacrylate, a polymer of 2-methoxyethyl acrylate, a polymer of 2-methoxyethyl methacrylate, and a polymer of glycidyl methacrylate. The cross-linked structure included in the shell portion is formed by using, for example, trimethylolpropane trimethacrylate.

In this regard, when the core portion according to the present embodiment has the cross-linked structure, the europium complex tends to remain in the core portion and does not readily leak outside the resin particle. The cross-linked structure included in the core portion is formed by using, for example, divinylbenzene.

In addition, since the core portion contains the copolymer having the unit denoted by Formula (CORE_1) above and since the polydispersity index is less than 0.1, in particular, the sensitivity is high when a target substance is detected by using a fluorescent depolarization method. The reason will be described below in detail.

### Measurement of Particle Size

In the present embodiment, the particle size (diameter) of the resin particle may be determined by a dynamic light scattering method. When a particle dispersed in a liquid is irradiated with laser light, and the scattered light is observed by using a photon detector, since the position of the particle continuously moves due to the Brownian motion, the intensity distribution due to interference of the scattered light continuously fluctuates.

Herein, the dynamic light scattering method is a measuring method for observing the manner of the Brownian motion as fluctuation of the scattered light intensity. The fluctuation of the scattered light over time is represented by an autocorrelation function, and a translational diffusion coefficient is determined. A Stokes diameter is determined from the resulting diffusion coefficient, and a particle size dispersed in a solution is derived. In addition, to express the width of the particle size distribution, the polydispersity index (PDI) is calculated by measurement, and the value of the polydispersity index being more than 0 and 0.1 or less indicates that the particle size distribution of the sample is monodisperse.

Herein, the fluorescent depolarization method has high detection sensitivity by detecting a change in the motion of the resin particle. In general, the rotational motion of a small particle is fast, and the rotational motion of a large particle is slow. In the present embodiment, the particle is aggregated due to an antigen-antibody reaction, the aggregated particle, for example, particle (antibody)-antigen-particle (antibody), becomes large, and slowing down of the rotational motion of the particle is detected.

Consequently, when a small particle and a large particle are present together in a system at an initial stage before the reaction, there is a concern that the detection sensitivity may deteriorate. Therefore, it is necessary that the resin particle have uniform particle size distribution. Specifically, it is required that the above-described polydispersity index is 0.1 or less.

The resin particle according to the present embodiment will be described below in detail.

### Structure of Resin Particle and Core-Shell Structure

In the present embodiment, the function of the resin particle is allotted between the core portion and the shell portion. To begin with, it is important that the resin material of the core portion has low specific gravity. It is assumed that the resin particle is used after being left to stand and stored for a long time of, for example, a several months.

Consequently, when the specific gravity of the particle is high, the resin particle settles in a container, steps of agitation and redispersion are required, and operations become complicated. Therefore, to suppress the resin particle from settling, a material having low specific gravity, more specifically, having specific gravity of 1.10 g/cm³ or less, can be used for the core portion that occupies most of the volume of a resin particle. Specifically, polystyrene can be used.

Next, regarding the resin particle of the shell portion, it is important to suppress particles from being aggregated and to suppress the particle from adsorbing to the container. Essentially, the inspection by using the fluorescent depolarization method is intended to detect aggregation of the particle, for example, particle (antibody)-antigen-particle (antibody), due to the antigen-antibody reaction. Therefore, simple aggregation of particle-particle and aggregation of a particle provided with an antibody and a specimen that does not react with the antibody have a bad influence of deteriorating the accuracy of the inspection. This action is referred to as nonspecific adsorption and is one of causes of deterioration of inspection sensitivity. Nonspecific adsorption between a particle and a particle (or specimen) may be caused by bonding of hydrophobic sections of the particle surfaces close to each other due to hydrophobic interaction. In this regard, when a long hydrophilic group is included on the resin surface, a particle and a particle (or specimen) may be aggregated due to entanglement of hydrophilic groups with each other.

Accordingly, the surface of a resin material for forming the shell portion according to the present embodiment is made to have a function of adsorbing a water molecule by a hydrogen bond at a short distance from the particle surface. Consequently, even when a particle and a particle (or specimen) approach each other, a water molecule being coordinated in a gap between the particles and suppressing particle surfaces from coming into direct contact with each other or suppressing entanglement at a long distance from occurring enables the particle and the particle (or specimen) to be prevented from being aggregated. Likewise, when a particle approaches a hydrophobic container, a water molecule being coordinated between the particle and the container enables the particle to be prevented from adhering to the container. In particular, the relationship between the radius (D1) of the core portion and the film thickness (D2) of the shell portion, illustrated in FIG. 1, can satisfy Formula (RA_1) below.

50 >D1/D2 >5/3 (RA_1)

When the cross-linked shell portion is thin, an effect of reducing nonspecific adsorption is not readily obtained. On the other hand, when the shell portion is thick, since the volume occupied by the shell portion in a resin particle increases, the specific gravity of the resin particle increases. As a result, unfavorably, the sedimentation velocity of the particle increases when the resin particle is stored.

### Europium Complex

In the present embodiment, a europium complex exhibiting polarization anisotropy is used based on the features that the wavelength and the intensity of light emission do not readily influenced by the surroundings and that the light emission has a long life. The europium complex 3 is composed of a europium element and a ligand. In the fluorescent depolarization method, the europium complex can be used in consideration of an emission lifetime, a visible emission wavelength region, and the like. In general, europium has an emission lifetime of 0.1 ms or more and 1.0 ms or less. The emission lifetime and a rotational relaxation time obtained from Formula (A1) described later have to be appropriately adjusted. Regarding the particle size of europium in a water dispersion solution, the diameter is preferably 80 nm or more and 200 nm or less since the polarization anisotropy denoted by Formula (A3) below largely changes between before and after an antigen-antibody reaction.

Of ligands constituting the europium complex 3, at least one ligand has a light collecting function. The light collecting function is an action to be excited at a specific wavelength and to excite a central metal of the complex by energy transfer. In addition, a ligand such as P-diketone can be present in the ligands constituting the europium complex 3 so as to prevent a water molecule from being coordinated. Since a ligand such as 3-diketone being coordinated with a rare earth ion suppresses a deactivation process due to energy transfer to a solvent molecule or the like, intense fluorescence emission is obtained.

The europium complex 3 may be a polynuclear complex provided that the polarization anisotropy is exhibited. The polarization anisotropy of the europium complex 3 is denoted by Formula (A3) described later. It is desirable that the polarization anisotropy be 0.08 or more in a state in which the rotational Brownian motion of the europium complex 3 in a medium is assumed to be stopped. The state in which the rotational Brownian motion is assumed to be stopped indicates a state in which the rotational relaxation time of a particle is sufficiently longer than the emission lifetime of the europium complex 3.

A larger amount of europium complex 3 can be taken in the core portion 1 since the emission intensity per particle is increased. Specifically, the content of the europium complex per g of particle is preferably 0.001 g or more.

In this regard, the content of the europium complex may be calculated by quantifying europium by using high-frequency inductively coupled plasma (ICP) emission spectrometry.

The europium complex in the resin particle according to the present embodiment is denoted by, for example, Formula (COMP_1) below.

Eu(A)ₓ(B)_{y}(C)_{z} (COMP_1)

Herein, in Formula (COMP_1), (A) is a ligand denoted by Formula (COMP_2) below, (B) is a ligand denoted by Formula (COMP_3) or Formula (COMP_4) below, and (C) is a ligand denoted by Formula (COMP_5) below.

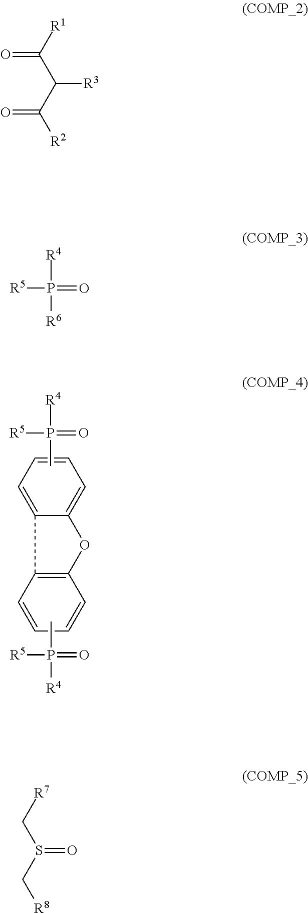

In Formula (COMP_2) to Formula (COMP_5), each of R1 and R2 represents an alkyl group, a perfluoroalkyl group, a phenyl group, or a thiophene group that may have a substituent, R3 represents a hydrogen atom or a methyl group, each of R4 and R5 represents an alkyl group or a phenyl group that may have a substituent, R6 represents an alkyl group, a phenyl group, or a triphenylene group that may have a substituent, each of R7 and R8 represents an alkyl group or a phenyl group that may have a substituent, in Formula (COMP_4), a bond indicated by a dotted line is not limited to being present, each substituent is any one of a methyl group, a fluoro group, a chloro group, and a bromo group, a carbon number of each alkyl group is 2 or more and 12 or less, and x, y, and z satisfy Formula (COMP_6), Formula (COMP_7), Formula (COMP_8), and Formula (COMP_9) below.

$$x = 3 ( COMP\_ 6 )$$

$$y = 1 or 2 ( COMP\_ 7 )$$

$$z = 0 or 1 ( COMP\_ 8 )$$

$$x + y + z = 4 or 5 ( COMP\_ 9 )$$

In addition, regarding the europium complex according to the present embodiment, a structure denoted by Formula (COMP_10) or Formula (COMP_11) below is exemplified.

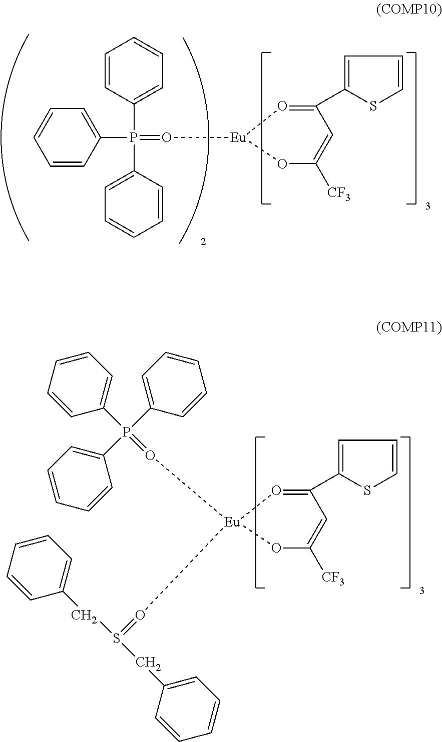

The structure denoted by Formula (COMP_10) may be expressed as Eu(TTA)₃(TPPO)₂ (Resin particles 1 to 5 and 7 to 15 in Examples and Comparative examples described later).

The structure denoted by Formula (COMP_11) may be expressed as Eu(TTA)₃(TPPO)(DBSO) (Resin particle 6 in Example described later).

On the other hand, the europium complex 3 being aggregated in the core portion 1 has an influence on the excitation efficiency and the like of the europium complex 3 due to an interaction between ligands, and measurement of the polarization anisotropy with reproducibility becomes difficult. Regarding a method for determining whether the europium complex 3 exhibits non-aggregation emission behavior in the core portion 1, determination may be performed by obtaining an excitation spectrum of a sample.

Since a light-emitting particle having high emission intensity is not only simply capable of being measured with high sensitivity but also capable of accelerating a biochemical reaction velocity since intense light emission is maintained even when a particle size is decreased. Since the diffusion coefficient of the Brownian motion increases with decreasing the particle size, the reaction is able to be detected in a shorter time (method for manufacturing resin particle).

### Core Portion and Radical Polymerizable Monomer

A radical polymerizable monomer serving as a material for forming the core portion contains at least a copolymer of a styrene-based monomer and styrenesulfonic acid as a main component. In the present embodiment, the main component is a material the mixing amount of which is the largest among resin materials constituting the core portion.

### Core Portion

When simple styrene is used as the main component for forming the core portion, the particle size is more than 500 nm and is unsuitable for the resin particle used for the fluorescent depolarization method. In this regard, a material having a charge being included during particle synthesis enables particles to be suppressed from coalescing and associating during particle synthesis since repulsion between particles due to the charge is utilized. Specifically, styrenesulfonic acid is useful as the material having a charge.

In the present embodiment, a copolymer with styrenesulfonic acid having a charge is necessary. However, the ratio of styrene to styrenesulfonic acid changes in accordance with selection of a polymerization initiator and, therefore, is not unconditionally determined. At least, the amount of styrene monomer has to be more than 10 times the amount of styrenesulfonic acid monomer. When styrenesulfonic acid is excessively added, a fine particle is generated, the polydispersity index deteriorates, and as a result, there is a concern that the detection sensitivity may deteriorate.

In this regard, a monomer selected from the group consisting of acrylate-based monomers and methacrylate-based monomers may be contained in addition to the main component. Examples include butadiene, vinyl acetate, vinyl chloride, acrylonitrile, methyl methacrylate, methacrylonitrile, and methyl acrylate. One type of these monomers may be used alone, or two or more types thereof may be used in combination.

Further, it is necessary that the resin material of the core portion is cross-linked by a monomer having two or more double bonds in the molecule, such as divinylbenzene, trimethylolpropane trimethacrylate, or ethylene glycol dimethacrylate. The measurement principle of the inspection by using fluorescent depolarization is to detect a change in the motion of the resin particle by a europium complex. Therefore, when the core portion is not cross-linked, the europium complex performs rotational motion in the interior of the resin particle. Accordingly, the motion of the resin particle and the motion of the europium complex are not readily linked to each other, and there is a concern that the detection sensitivity may deteriorate. On the other hand, when the core portion is cross-linked, since the motion of the europium complex in the resin particle is suppressed, it is possible to improve the detection sensitivity.

In this regard, the europium complex obtains the emission intensity due to a ligand. Therefore, when the ligand is exposed to the environment outside the particle and is removed, there is a concern that the emission intensity deteriorates.

In the present embodiment, the core portion being cross-linked enables the europium complex to remain in the core portion and enables the ligand to be suppressed from being removed due to exposure so as to cause deterioration of the emission intensity.

In particular, divinylbenzene can be used for a cross-linking agent of the core portion. Since divinylbenzene is a compound analogous to styrene that is a structure of the main component of the core portion, divinylbenzene is capable of uniformly spreading in the core portion without being localized in the core portion. Accordingly, uniform cross-linking may occur in the core portion. As a result, cross-linking enables the europium complex to be suppressed from performing the rotational motion and enables the europium complex to be suppressed from being exposed outside the resin particle.

Presence or absence of cross-linking may be determined by the following method.

After the resin particle is dispersed in pyridine at a concentration of 5% by weight, shaking is performed at 50° C. for 3 hours. Before and after this operation, the particle size is measured by the above-described dynamic light scattering method. When cross-linking is not performed, the resin particle is dissolved, and measurement is difficult.

Alternatively, particles are coagulated together, the particle size becomes twice or more the particle size before the operation, and the shape of the particle is not maintained.

### Shell Portion

The main components of the shell portion are resins denoted by Formula (SHELL_1) and (SHELL_2) below.

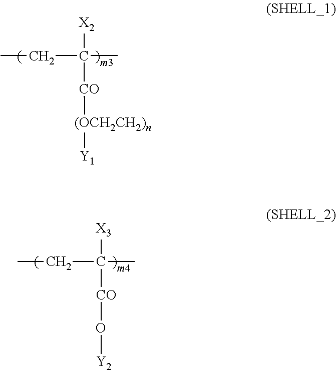

In Formula (SHELL_1) and Formula (SHELL_2), each of X₂ and X₃ represents H or CH₃, Y₁ represents OH or OCH₃, Y₂ represents Formula (SHELL_3) below or CH₂CH₂OH, each of m3 and m4 represents an integer of 1 or more, and n represents an integer of 1 or more and 40 or less.

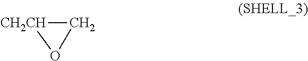

Herein, the main component is a material the mixing amount of which is the largest among resin materials constituting the shell portion. Regarding the material for forming the shell portion, a hydrogen bond section is arranged close to a main chain of a side chain. The hydrogen bond section is capable of forming a hydrogen bond with a water molecule in the system. The water molecule is fixed in the vicinity of the shell. According to the water molecule, even when another resin particle, a specimen that does not cause the antigen-antibody reaction, an inspection container, or the like approaches the resin particle, the water molecule being coordinated in a gap between the particles enables particle surfaces to be suppressed from coming into direct contact with each other. According to this action, it is possible to suppress nonspecific adsorption between resin particles and between a resin particle and a specimen from occurring.

Regarding the material for forming the shell portion, specifically, it is favorable that the shell portion be composed by using hydroxyethyl methacrylate, polyethylene glycol monomethyl ether methacrylate, 2-methoxyethyl acrylate, 2-methoxyethyl methacrylate, and glycidyl methacrylate as polymerizable monomers denoted by Formulae (SHELL_4), (SHELL_5), and (SHELL_6) below and performing polymerization outside the core portion.

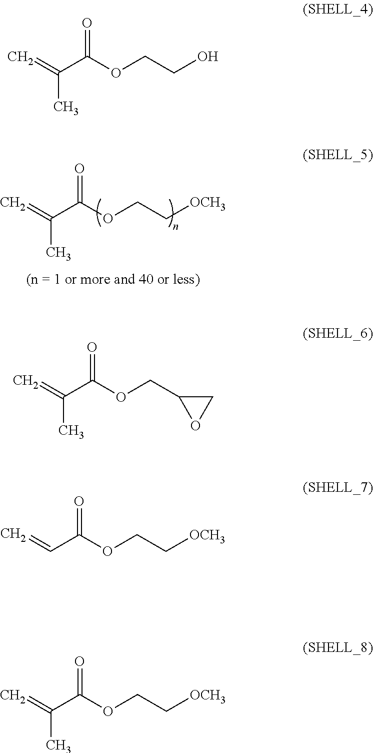

### Shell Portion Cross-Linking

In addition, the resin material for forming the shell portion has to be cross-linked by a monomer having two or more double bonds in the molecule, such as divinylbenzene, trimethylolpropane trimethacrylate, or ethylene glycol dimethacrylate.

FIG. 2 is a schematic diagram illustrating a resin particle in the related art in which a non-cross-linked hydrophilic polymer 20 is adsorbed to the surface of the core portion 1. The non-cross-linked hydrophilic polymer 20 being adsorbed to the surface of the resin particle enables nonspecific adsorption of the resin particle to be reduced from occurring, but the following disadvantage is caused. The core portion 1 includes a europium complex 3. In FIG. 2, a region E2 in which the hydrophilic polymer 20 covers the surface of the core portion 1 and a region E1 at which the surface of the core portion is exposed are present together. The reason for this is considered to be that since the surface composition of the core portion is composed of a fine hydrophobic region and a fine hydrophilic region, a hydrophilic polymer readily adsorbs to a hydrophilic surface of the core portion, whereas a hydrophilic polymer does not readily adsorb to a hydrophobic surface of the core portion.

When a resin particle in the related art is placed in an inspection container, the resin particle adsorbs to the surface of the inspection container due to a hydrophobic interaction between the surface of the inspection container not having hydrophilicity and the hydrophobic portion (E1 in FIG. 2) of the surface of the resin particle. The motion of the resin particle is restricted since the resin particle adsorbs to the container, and as a result, a disadvantage that the sensitivity of the fluorescent depolarization method deteriorates occurs.

On the other hand, in the present embodiment, as illustrated in FIG. 1, since the resin of the shell portion is cross-linked, regarding the hydrophobic surface of the core portion, the shell portion material is forcedly fixed to the particle surface. As a result, the particle surface of the core portion is uniformly covered with the shell portion material.

In addition, the shell portion being cross-linked enables a hydrogen bond section arranged in the side chain of the shell portion material to be prevented from widely extending toward water serving as a solvent. When the shell portion is not cross-linked, the hydrophilic polymer spreads toward the water, and there is a concern of an interaction with a hydrophilic section of another particle (or specimen) that approaches the hydrophilic polymer. On the other hand, in the present embodiment, since the shell member is cross-linked, the shell portion material is not separated from the resin particle so as to spread. Therefore, since an interaction with another particle (or specimen) that approaches the hydrophilic polymer does not readily occur, it is possible to suppress particles from adsorbing to each other and to improve the nonspecific adsorbability.

In particular, trimethylolpropane trimethacrylate can be used as a cross-linking agent of the shell portion. Trimethylolpropane trimethacrylate is a compound analogous to the structure of the main component of the shell portion and, therefore, is capable of uniformly spreading over the shell portion without being localized in the shell portion. Consequently, it is possible to perform uniform cross-linking of the entire shell portion and, as a result, to improve the nonspecific adsorbability.

Presence or absence of cross-linking may be determined by the following method akin to that of the core portion.

After the resin particle is dispersed in pyridine at a concentration of 5% by weight, shaking is performed at 50° C. for 3 hours. Before and after this operation, the particle size is measured by the above-described dynamic light scattering method. When cross-linking is not performed, the resin particle is dissolved, and measurement is difficult. Alternatively, particles are coagulated together, the particle size becomes twice or more the particle size before the operation, and the shape of the particle is not maintained.

### Radical Polymerization Initiator

Regarding the radical polymerization initiator, azo compounds, organic peroxides, and the like may be widely used. Specific examples include 2,2′-azobis(isobutyronitrile), 2,2′-azobis(2,4-dimethylvaleronitrile), 2,2′-azobis(2-methylbutyronitrile), 4,4′-azobis(4-cyanovaleric acid), 2,2′-azobis(2-methylpropionamidine)dihydrochloride, dimethyl 2,2′-azobis(2-methylpropionate), tert-butyl hydroperoxide, benzoyl peroxide, ammonium persulfate (APS), sodium persulfate (NPS), and potassium persulfate (KPS).

### Water-Based Medium

A buffer solution may be used as a water solvent. In this regard, to increase the stability of a liquid in which the resin particle according to the present embodiment is dispersed, a surfactant, a preservative, a sensitizer, and the like may be added to the water solvent.

The liquid in which the resin particle according to the present embodiment is dispersed being used enables the anisotropy of the polarized light emission with respect to the behavior of aggregation or dispersion of the particle to be detected with high sensitivity. Therefore, a colloidal liquid in which the resin particle according to the present embodiment is dispersed in a water solvent may be utilized as an inspection reagent having high sensitivity when the fluorescent depolarization method is used.

### Ligand and Affinity Particle

The present embodiment provides an affinity particle including the resin particle according to the present embodiment and a ligand. In such an instance, the shell portion of the resin particle has at least one reactive functional group selected from the group consisting of a carboxy group, an amino group, a thiol group, an epoxy group, a maleimide group, and a succinimidyl group. The ligand is bonded to the functional group. The reactive functional group is included on the surface side of the resin particle, that is, on the opposite side of the center.

In the present embodiment, the ligand is a compound that is specifically bonded to a specific target substance. The section of the ligand that is bonded to the target substance is always the same and has selectively or specifically high affinity. Examples include an antigen and an antibody, an enzyme protein and a substrate thereof, a signal substance such as a hormone or a neurotransmitter and a receptor thereof, and a nucleic acid. The ligand according to the present embodiment is not limited to these. Examples of the nucleic acid include deoxyribonucleic acid. In the present embodiment, the affinity particle is a particle having selectively or specifically high affinity for a target substance. The ligand according to the present embodiment can be any one of an antibody, an antigen, and a nucleic acid.

In the present embodiment, regarding the chemical reaction to chemically bond the reactive functional group included in the resin particle according to the present embodiment to the ligand, a known method in the related art may be applied within the bounds of exerting the effect of the present disclosure. When the ligand is bonded by an amide bond, a catalyst such as 1-[3-(dimethylaminopropyl)-3-ethylcarbodiimide]may be appropriately used. The resin particle according to the present embodiment may fix the ligand by physical adsorption.

When an antibody (antigen) is used as the ligand, and an antigen (antibody) is used as the target substance, the affinity particle according to the present embodiment can be applied to an immune latex agglutination method and a fluorescent depolarization method.

### In Vitro Diagnosis Inspection Reagent

In the present embodiment, an in vitro diagnosis inspection reagent, that is, an inspection reagent used to detect a target substance in a specimen during in vitro diagnosis contains the affinity particle according to the present embodiment and a dispersion medium to disperse the affinity particle. In the present embodiment, the amount of the affinity particle according to the present embodiment contained in the inspection reagent is preferably 0.000001% by mass to 20% by mass and more preferably 0.0001% by mass to 1% by mass. The inspection reagent according to the present embodiment may contain a third substance such as a solvent or a blocking agent in addition to the affinity particle according to the present embodiment within the bounds of exerting the effect of the present disclosure. Two or more types of the third substances such as a solvent and a blocking agent may be contained in combination. Examples of the solvent used in the present embodiment include various buffer solutions, such as phosphate buffer solutions, glycine buffer solutions, Good's buffer solutions, Tris buffer solutions, and ammonia buffer solutions. The solvent contained in the inspection reagent according to the present embodiment is not limited to these.

When the inspection reagent according to the present embodiment is used to detect an antigen or an antibody in a specimen, an antibody or an antigen may be used as the ligand.

### Inspection Kit

In the present embodiment, an inspection kit used to detect a target substance in a specimen during in vitro diagnosis includes the above-described reagent and a casing including the above-described reagent. The kit according to the present embodiment may contain a sensitizer to facilitate aggregation of the particle during the antigen-antibody reaction. Examples of the sensitizer include polyvinyl alcohols, polyvinylpyrrolidones, and sodium alginate, but the present disclosure is not limited to these. In addition, the inspection kit according to the present embodiment may be provided with positive control, negative control, a blood serum diluent, and the like. As a medium of the positive control and the negative control, blood serum containing no measurable target substance, a physiological saline solution, and, in addition, a solvent may be used. The inspection kit according to the present embodiment may be used for a method for detecting the target substance according to the present embodiment in the manner akin to that of a common kit used to detect a target substance in a specimen during in vitro diagnosis. In this regard, the concentration of the target substance may be measured by a known method in the related art, and the inspection kit is suitably used to detect the target substance in a specimen by the immune latex agglutination method or the fluorescent depolarization method.

### Detection Method

A method for detecting a target substance in a specimen during in vitro diagnosis according to the present embodiment includes a step of mixing the affinity particle according to the present embodiment and a specimen having possibility of containing a target substance. In this regard, mixing of the affinity particle according to the present embodiment and the specimen can be performed within a pH range of 3.0 to 11.0. In addition, the mixing temperature is within the range of 20° C. to 50° C., and the mixing time is within the range of 1 min to 60 min. The present detection method can use a solvent. In the detection method according to the present embodiment, the concentration of the affinity particle according to the present embodiment in the reaction system is preferably 0.000001% by mass to 1% by mass and more preferably 0.00001% by mass to 0.001% by mass. Regarding the method for detecting a target substance in a specimen according to the present embodiment, an aggregation reaction caused as a result of mixing of the affinity particle according to the present embodiment and the specimen can be detected by the fluorescent depolarization method. Specifically, a step of obtaining a liquid mixture by mixing the specimen into the inspection reagent, a step of applying polarized light to the liquid mixture, and a step of separating and detecting a polarization component in the light emission from the affinity particle in the liquid mixture.

The above-described aggregation reaction caused in the liquid mixture being optically detected enables the target substance in the specimen to be detected and further enables the concentration of the target substance to be measured.

### Fluorescent Depolarization Method

A europium complex that emits polarized light being included as a material in the resin particle enables polarized light emission characteristics to be grasped even when a dispersion state of the particle in the liquid is slightly changed. Specifically, when the antigen-antibody reaction occurs and particles are aggregated with an antigen interposed therebetween, it is possible to grasp a change in the rotational Brownian motion of the particle as a change in the polarization anisotropy.

The polarization anisotropy means that a transition moment (transition dipole moment) has anisotropy. In general, regarding a light-emitting coloring material exhibiting a transition moment having anisotropy, polarized light emission means that, when polarized light along the transition moment is assumed to be excitation light, light emission is also polarized along the transition moment. Regarding the europium complex, since fluorescence emission based on energy transfer from the ligand to a central metal ion is exhibited, the transition moment of the polarized light emission becomes complicated, and red light emission in the vicinity of 610 nm derived from electron transition from the lowest excitation state 5D0 to 7F2 has polarization anisotropy.

The principle of the fluorescent depolarization method is to measure a shift of the transition moment due to the rotational motion of the light-emitting material in a time in which the polarized light emission occurs. The rotational motion of the light-emitting material is denoted by Formula (A1).

$$Q = 3 V η / kT \qquad ( A1 )$$

Herein,
Q: rotational relaxation time of material
V: volume of material
f: viscosity of solvent
k: Boltzmann constant
T: absolute temperature.

The rotational relaxation time of the material is a time necessary for the molecule to rotate an angle of θ(68.5°) at which cos θ=1/e.

It is clear from Formula (A1) that the rotational relaxation time of the light-emitting material is in proportion to the volume of the material, that is, the cube of the particle radius. On the other hand, regarding fluorescent depolarization, the relationship between the emission lifetime and the degree of polarization of the material is denoted by Formula (A2).

$$p 0 / p = 1 + A ( τ / Q ) \qquad ( A2 )$$

Herein,
p0: degree of polarization when material is stopped (Q=∞)
p: degree of polarization
A: constant
τ: emission lifetime of material
Q: rotational relaxation time.

According to Formula (A1) and Formula (A2), to obtain a large change in measurement of the degree of polarization, the relationship between the emission lifetime of the light-emitting material and the rotational relaxation time, that is, the volume (particle size) of the light-emitting material, is important, and the emission lifetime has to be increased with increasing the particle size of the light-emitting material.

When the degree of polarization of the light emission denoted by Formula (A2) is determined from an experiment, polarized light may be incident on a sample, and light emission may be detected in the direction perpendicular to the forward direction and the vibration direction of the excitation light. In such an instance, the detected light may be separated into polarization components in the directions parallel and perpendicular to the polarized light of the incident light and the degree of polarization may be evaluated by using a numerical expression denoted by Formula (A3).

$$r ( t ) = ( I / / ( t ) - GI ⊥ ( t ) ) / ( I / / ( t ) + 2 GI ⊥ ( t ) ) \qquad ( A3 )$$

Herein,
r(t): polarization anisotropy at time t
I//(t): emission intensity of emission component parallel to excitation light at time t
I⊥(t): emission intensity of emission component perpendicular to excitation light at time t
G: correction value, ratio of IL/I// measured by using excitation light having vibration direction 90-degree different from that of excitation light used for sample measurement. That is, in a range of appropriate particle size and emission lifetime, it is possible to keenly read a change in the particle size of the light-emitting material due to the antigen-antibody reaction or the like as a value of polarization anisotropy. In this regard, the polarization anisotropy is a value of the degree of polarization corrected by G and 2G, and the degree of polarization is a value of Formula (A3) where G and 2G are removed. In the actual measurement, since a correction value of G is necessary, the polarization anisotropy is determined.

Further, regarding the light-emitting particle according to the present embodiment, the polarization anisotropy “r” determined by Formula (A4) below can be 0.01 or more.

$$r = \frac{{I}_{VV} - G * {I}_{VH}}{{I}_{VV} + 2 * G * {I}_{VH}} \qquad ( A4 )$$

$$G = \frac{{I}_{HV}}{{I}_{HH}}$$

In Formula (A4),
r polarization anisotropy
I_{VV} emission intensity of emission component having vibration direction parallel to that of first polarized light when excitation is caused by first polarized light
I_{VH} emission intensity of emission component having vibration direction orthogonal to that of first polarized light when excitation is caused by first polarized light
I_{HV} emission intensity of emission component having vibration direction orthogonal to that of second polarized light when excitation is caused by second polarized light having vibration direction orthogonal to that of first polarized light
I_{HH} emission intensity of emission component having vibration direction parallel to that of second polarized light when excitation is caused by second polarized light having vibration direction orthogonal to that of first polarized light
G correction value.

### EXAMPLES

The present disclosure will be specifically described below with reference to Examples. However, the present disclosure is not limited to such Examples.

### (1) Production of Resin Particle 1

In a four-neck flask,
100 g of MES (2-morpholinoethanesulfonic acid) buffer solution having a pH of 7 (produced by KISHIDA CHEMICAL Co., Ltd.),
0.04 g of polarized light emission europium complex [tris(2-thenoyltrifluoroacetonato)bis(triphenylphosphineoxide)europium(III)](hereafter abbreviated as “Eu(TTA)₃(TPPO)₂”, produced by Central Techno Corporation),
1.00 g of styrene monomer (produced by KISHIDA CHEMICAL Co., Ltd.),
0.10 g of sodium styrenesulfonate (styrenesulfonic acid monomer) (produced by TOKYO KASEI KOGYO CO., LTD.), and
0.13 g of divinylbenzene (hereafter abbreviated as (DVB)) (produced by TOKYO KASEI KOGYO CO., LTD.)
   were agitated where a mechanical stirrer was set at 300 rpm. After agitation was performed for 15 min under a nitrogen flow condition, the temperature of an oil bath prepared in advance was set to be 70° C., and nitrogen flow was further performed for 15 min. After the liquid mixture was heat-agitated, 0.01 g of 2,2′-azobis(2-methylpropionamidine)dihydrochloride (hereafter abbreviated as “V50”) (produced by FUJIFILM Wako Pure Chemical Corporation) was added, and emulsion polymerization was performed for 6 hours so as to produce a core portion.

After the polymerization reaction, the obtained suspension solution was mixed with
0.20 g of hydroxyethyl methacrylate (hereafter abbreviated as “HEMA”) (produced by TOKYO KASEI KOGYO CO., LTD.),
0.05 g of trimethylolpropane trimethacrylate (hereafter abbreviated as “TMP”) (produced by TOKYO KASEI KOGYO CO., LTD.), and
0.01 g of V50
   serving as materials for forming a shell portion, and emulsion polymerization was performed for 10 hours so as to produce the shell portion.

After the polymerization reaction, the obtained suspension solution was subjected to ultrafiltration by using an ultrafilter with molecular weight cut-off of 100 K and about 4L of ion-exchanged water, and the product was washed so as to obtain a dispersion solution of Resin particle 1. The amount of each component used to produce Resin particle 1 is presented in Table 1.

(2) Production of Resin particles 2 to 15

The composition ratios of reagents used to produce Resin particles 2 to 15 in the same procedure are presented in Table 1. In this regard, the materials used therefor are as described below.
[tris(2-thenoyltrifluoroacetonato)(triphenylphosphineoxide)(dibenzylsulfoxide)europium(III)](hereafter abbreviated as “Eu(TTA)₃(TPPO)(DBSO)”, produced by Central Techno Corporation),
ethylene glycol dimethacrylate (produced by TOKYO KASEI KOGYO CO., LTD.),
polyethylene glycol monomethyl ether methacrylate (hereafter abbreviated as “PEG”) (produced by Sigma-Aldrich Japan),
glycidyl methacrylate (hereafter abbreviated as “GMA”) (produced by TOKYO KASEI KOGYO CO., LTD.),
2-methoxyethyl acrylate (hereafter abbreviated as “MEA”) (produced by TOKYO KASEI KOGYO CO., LTD.), and
2-methoxyethyl methacrylate (hereafter abbreviated as “MEMe”) (produced by TOKYO KASEI KOGYO CO., LTD.)

TABLE 1

| | | | | Core portion material | | | | |
|---|---|---|---|---|---|---|---|---|
| | MES | | | | | Styrene/ | | |
| | buffer | Europium | | Styrene | Styrenesulfonic | styrenesulfonic | Core cross-linking | |
| | solution | complex | | monomer | acid monomer | acid [mol/mol] | agent | |
| Resin particle 1 | 100 g | Eu(TTA)3 | 0.04 g | 1.00 g | 0.10 g | 19.80 | DVB | 0.13 g |
| | | (TPPO)2 | | | | | | |
| Resin particle 2 | 100 g | Eu(TTA)3 | 0.04 g | 1.00 g | 0.10 g | 19.80 | DVB | 0.13 g |
| | | (TPPO)2 | | | | | | |
| Resin particle 3 | 100 g | Eu(TTA)3 | 0.04 g | 1.00 g | 0.10 g | 19.80 | DVB | 0.13 g |
| | | (TPPO)2 | | | | | | |
| Resin particle 4 | 100 g | Eu(TTA)3 | 0.04 g | 1.00 g | 0.10 g | 19.80 | DVB | 0.13 g |
| | | (TPPO)2 | | | | | | |
| Resin particle 5 | 100 g | Eu(TTA)3 | 0.04 g | 1.00 g | 0.10 g | 19.80 | DVB | 0.13 g |
| | | (TPPO)2 | | | | | | |
| Resin particle 6 | 100 g | Eu(TTA)3 | 0.04 g | 1.00 g | 0.10 g | 19.80 | DVB | 0.13 g |
| | | (TPPO) | | | | | | |
| | | (DBSO) | | | | | | |
| Resin particle 7 | 100 g | Eu(TTA)3 | 0.04 g | 1.00 g | 0.10 g | 19.80 | DVB | 0.13 g |
| | | (TPPO)2 | | | | | | |
| Resin particle 8 | 100 g | Eu(TTA)3 | 0.04 g | 1.00 g | 0.10 g | 19.80 | ethylene glycol | 0.13 g |
| | | (TPPO)2 | | | | | dimethacrylate | |
| Resin particle 9 | 100 g | Eu(TTA)3 | 0.04 g | 1.00 g | 0.07 g | 28.28 | DVB | 0.13 g |
| | | (TPPO)2 | | | | | | |
| Resin particle 10 | 100 g | Eu(TTA)3 | 0.04 g | 1.00 g | 0.09 g | 23.10 | DVB | 0.13 g |
| | | (TPPO)2 | | | | | | |
| Resin particle 11 | 100 g | Eu(TTA)3 | 0.04 g | 1.00 g | 0.10 g | 19.80 | DVB | 0.13 g |
| | | (TPPO)2 | | | | | | |
| Resin particle 12 | 100 g | Eu(TTA)3 | 0.04 g | 1.00 g | 0.10 g | 19.80 | DVB | 0.13 g |
| | | (TPPO)2 | | | | | | |
| Resin particle 13 | 100 g | Eu(TTA)3 | 0.04 g | 1.00 g | 0.10 g | 19.80 | DVB | 0.13 g |
| | | (TPPO)2 | | | | | | |
| Resin particle 14 | 100 g | Eu(TTA)3 | 0.04 g | 1.00 g | 0.26 g | 7.92 | DVB | 0.13 g |
| | | (TPPO)2 | | | | | | |
| Resin particle 15 | 100 g | Eu(TTA)3 | 0.04 g | 1.00 g | — | — | DVB | 0.13 g |
| | | (TPPO)2 | | | | | | |

| | Core portion | Shell portion material | | | | |
|---|---|---|---|---|---|---|
| | material | | | Shell cross-linking | | |
| | V50 | Monomer material | | agent | | V50 |
| Resin particle 1 | 0.01 g | HEMA | 0.20 g | TMP | 0.05 g | 0.01 g |
| Resin particle 2 | 0.01 g | PEG | 0.20 g | TMP | 0.05 g | 0.01 g |
| Resin particle 3 | 0.01 g | GMA | 0.20 g | TMP | 0.05 g | 0.01 g |
| Resin particle 4 | 0.01 g | MEA | 0.20 g | TMP | 0.05 g | 0.01 g |
| Resin particle 5 | 0.01 g | MEMe | 0.20 g | TMP | 0.05 g | 0.01 g |
| Resin particle 6 | 0.01 g | HEMA | 0.20 g | TMP | 0.05 g | 0.01 g |
| Resin particle 7 | 0.01 g | Polyethylene | 0.20 g | TMP | 0.05 g | 0.01 g |
| | | GlycolMono- | | | | |
| | | methacrylate | | | | |
| Resin particle 8 | 0.01 g | HEMA | 0.20 g | ethylene glycol | 0.05 g | 0.01 g |
| | | | | dimethacrylate | | |
| Resin particle 9 | 0.01 g | HEMA | 0.10 g | TMP | 0.05 g | 0.01 g |
| Resin particle 10 | 0.01 g | HEMA | 0.50 g | TMP | 0.05 g | 0.01 g |
| Resin particle 11 | 0.01 g | HEMA | 0.20 g | TMP | 0.05 g | 0.01 g |
| Resin particle 12 | 0.01 g | — | — | — | | 0.01 g |
| Resin particle 13 | 0.01 g | HEMA | 0.20 g | — | | 0.01 g |
| Resin particle 14 | 0.01 g | HEMA | 0.20 g | TMP | 0.05 g | 0.01 g |
| Resin particle 15 | 0.01 g | HEMA | 0.20 g | TMP | 0.05 g | 0.01 g |

Dispersion solutions of Resin particles 1 to 15 were obtained by the above-described emulsion polymerization.

Production of Affinity Particle Modified with Anti-CRP Antibody

A portion of HEMA was provided with a carboxylic acid group by replacing a dispersion solution of synthesized Resin particle 11 with pyridine and, thereafter, adding succinic anhydride.

After 0.25 mL of the dispersion solution of Resin particle 11 (1.2% by weight) provided with carboxylic acid was taken, the solvent was replaced with 1.6 mL of MES buffer solution having a pH of 6.0. The MES buffer solution including Resin particle 11 was mixed with 0.5% by weight of 1-[3-(dimethylamino)propyl]-3-ethylcarbodiimide and N-hydroxysulfosuccinimide sodium salt, and a reaction was performed at 25° C. for 1 hour. After the reaction, the dispersion solution was washed with the MES buffer solution having a pH of 5.0, 100 μg/mL of anti-CRP antibody was added, and the anti-CRP antibody was bonded to Resin particle 11 at 25° C. for 2 hours. After bonding, Resin particle 11 to which the antibody was bonded was washed with the Tris buffer solution having a pH of 8. After the reaction, Resin particle 11 to which the antibody was bonded was washed with the phosphate buffer solution so as to obtain an affinity particle modified with 0.3% by weight of concentration of anti-CRP antibody (hereafter also abbreviated as affinity particle).

The antibody being bonded to the resin particle was ascertained by measuring the amount of decrease of the antibody concentration in the buffer solution to which the antibody was added by using the BCA assay.

### Examples and Evaluation

Each evaluation was performed by using the prepared dispersion solution of the resin particle. Examples and the evaluation results are presented in Table 2. Evaluation method

### Particle Size, Polydispersity Index, Core Portion Radius, and Shell Portion Thickness

The shape of the resin particle was evaluated by using an electron microscope (S5500 produced by Hitachi High-Technologies Corporation). The average particle size of the resin particle was evaluated by using dynamic light scattering (ZETASIZER NANO S produced by Malvern). In addition, after the core portion was synthesized, the particle size of the core portion was measured by taking a portion of the dispersion solution of the resin particle and performing measurement by using dynamic light scattering. Further, after the shell portion was synthesized, the thickness of the shell portion was measured by taking a portion and performing measurement in the same manner. The polydispersity index is also obtained by the above-described measurement.

### Resin Particle Concentration and Eu Complex Concentration

The particle concentration in the suspension solution in which the resin particle was dispersed was evaluated by using a weight analyzer (Thermo Plus TG8120 produced by Rigaku Corporation). In addition, the amount of included Eu complex per g of the particle was measured by quantifying Eu by using ICP spectrometer (PS3510 produced by Hitachi High-Tech Corporation).

### Polarization Anisotropy and Nonspecific Adsorbability

A fluorescence spectrum of the resin particle was measured where the excitation light was 340 nm, and a polarizer was disposed in an excitation side optical path and a light-emission side optical path. The measurement was performed while the direction of the excitation side polarizer was fixed, and the direction of the light-emission side polarizer was set to be parallel or perpendicular to the direction of the excitation side polarizer. Regarding the apparatus, Fluorescence Spectrophotometer F-4500 produced by Hitachi High-Tech Science Corporation was used. The peak wavelength of the observation light to analyze the polarized light emission was set to be 611 nm. The obtained data of the polarized light emission was analyzed by using Formula (A4) above so as to determine the polarization anisotropy r. Subsequently, 10 μL of each resin particle dispersion solution (0.1 mg/mL) was added to 500 μL of human serum solution diluted with 15 times the amount of buffer solution, the temperature was kept at 37° C. for 10 min, and an amount of change Δr in the value of polarization anisotropy r between before and after the keeping of temperature was calculated. When the nonspecific adsorbability is high, since aggregation of the particle occurs, Δr increases. The nonspecific adsorbability can be decreased. Therefore, Δr can be close to zero.

Regarding the nonspecific adsorbability, Δr of 0.010 or less was rated as A, 0.012 or less was rated as B, and more than 0.012 was rated as C. The evaluation result of A or B was good, and C was unacceptable.

Particle Size after Storage at 40° C.

The produced resin particle dispersion solution was diluted with pure water and adjusted to 0.1 mg/mL. Thereafter, 8 mL of the dispersion solution was placed in a sealed container having a volume of 10 mL and stored at 40° C. for 3 months. A change in the particle size between before and after storage was measured by using dynamic light scattering.

The amount of change in the particle size of 7 nm or less was rated as A, 10 nm or less was rated as B, and more than 10 nm was rated as C. The evaluation result of A or B was good, and C was unacceptable.

Settleability after Storage while Leaving to Stand at 5° C.

The produced resin particle dispersion solution was diluted with pure water and adjusted to 0.1 mg/mL. Thereafter, 25 mL of the dispersion solution was placed in a sealed container having a volume of 30 mL and stored at 5° C. for 3 months. Before storage, 1 mL of the dispersion solution was taken from the upper portion in the container, and the absorbance at 527 nm was measured (UH5200 produced by Hitachi High-Tech Corporation). After storage, 1 mL of the dispersion solution was taken from the upper portion in the container, and the absorbance was measured in the same manner. The settleability of the resin particle was evaluated in accordance with the amount of change between the absorbance before storage and the absorbance after storage. When the settleability of the resin particle is good, that is, when the settlement is suppressed, the amount of change in the absorbance is a small value.

The amount of change in the absorbance of 0.02 or less was rated as A, 0.03 or less was rated as B, and more than 0.03 was rated as C. The evaluation result of A or B was good, and C was unacceptable.

### Evaluation of Affinity Particle

Regarding the obtained affinity particle, the polarization anisotropy was measured before and after mixing with CRP (antigen). The concentration of the affinity particle was fixed to 0.0001 mg/mL, investigation was performed at the CRP concentration of 0 to 10,000 μg/mL.

Regarding the evaluation, a microplate reader (Nivo produced by PerkinElmer, Inc.) was used. A filter with a central wavelength of 355 nm and a half-value width of 40 nm was used as an excitation light filter, a filter with a central wavelength of 615 nm and a half-value width of 8 nm was used as a light emission filter, and D400 was used as a dichroic mirror. The measurement time was set to be 1 sec, the polarization anisotropy r in 30 min from start of the reaction was measured, and the amount of change Δr in the polarization anisotropy r during the measurement was calculated. The measurement temperature was fixed to 37° C. The evaluation results are presented in Table 3.

TABLE 2

| | | Content of | | | | | Cure | Evaluation result | | |
|---|---|---|---|---|---|---|---|---|---|---|
| | | europlum | | | | | radius/ | Δr | | Settleability |
| | | complex per | Poly- | | | Shell | shell | Nonspecific | Particle size | after storage |
| | | g of resin | dispersity | Particle | Core | thick- | thick- | adsorb- | after storage | while leaving |
| | Resin particle | particle | index | size | radius | ness | ness | ability | at 40° C. | to stand at 5° C. |
| Example 1 | Resin particle 1 | 0.004 [g/g] | 0.05 | 120 nm | 50 nm | 10 nm | 5.00 | A | A | A |
| Example 2 | Resin particle 2 | 0.004 [g/g] | 0.05 | 120 nm | 50 nm | 10 nm | 5.00 | A | A | A |
| Example 3 | Resin particle 3 | 0.004 [g/g] | 0.05 | 120 nm | 50 nm | 10 nm | 5.00 | A | A | A |
| Example 4 | Resin particle 4 | 0.004 [g/g] | 0.05 | 120 nm | 50 nm | 10 nm | 5.00 | A | A | A |
| Example 5 | Resin particle 5 | 0.004 [g/g] | 0.05 | 120 nm | 50 nm | 10 nm | 5.00 | A | A | A |
| Example 6 | Resin particle 6 | 0.004 [g/g] | 0.05 | 120 nm | 50 nm | 10 nm | 5.00 | A | A | A |
| Example 7 | Resin particle 7 | 0.004 [g/g] | 0.05 | 120 nm | 50 nm | 10 nm | 5.00 | A | B | A |
| Example 8 | Resin particle 8 | 0.004 [g/g] | 0.05 | 120 nm | 50 nm | 10 nm | 5.00 | A | B | A |
| Example 9 | Resin particle 9 | 0.004 [g/g] | 0.08 | 183 nm | 50 nm | 1.5 nm | 60.00 | B | A | A |
| Example 10 | Resin particle 10 | 0.004 [g/g] | 0.07 | 190 nm | 50 nm | 40 nm | 1.38 | A | A | B |
| Example 11 | Resin particle 11 | 0.004 [g/g] | 0.05 | 120 nm | 50 nm | 10 nm | 5.00 | A | A | A |
| Comparative | Resin particle 12 | 0.004 [g/g] | 0.05 | 100 nm | 50 nm | — | — | C | C | B |
| example 1 | | | | | | | | | | |
| Comparative | Resin particle 13 | 0.004 [g/g] | 0.08 | 130 nm | 50 nm | 15 nm | 3.33 | C | B | B |
| example 2 | | | | | | | | | | |
| Comparative | Resin particle 14 | 0.004 [g/g] | 0.15 | 180 nm | 70 nm | 20 nm | 3.50 | C | B | C |
| example 3 | | | | | | | | | | |
| Comparative | Resin particle 15 | 0.004 [g/g] | 0.08 | 260 nm | 110 nm | 20 nm | 5.50 | C | B | C |
| example 4 | | | | | | | | | | |

According to the results presented in Table 2, as indicated by comparison between Comparative examples and Examples, it was found that the shell portion being included, the shell portion having a cross-linked structure, the polydispersity index being 0.1 or less, and the core portion containing a specific copolymer enable nonspecific adsorption to be reduced.

TABLE 3

| | CRP antigen concentration pg/mL | | | | | |
|---|---|---|---|---|---|---|
| | 1 | 5 | 10 | 100 | 1000 | 10000 |
| Amount of | 0.0041 | 0.0044 | 0.0052 | 0.0060 | 0.0064 | 0.0102 |
| change in | | | | | | |
| polarization | | | | | | |
| anisotropy Δr | | | | | | |

According to the results presented in Table 3, it is ascertained that the amount of change (Δr) in the polarization anisotropy between before and after mixing with CRP was changed in accordance with the CRP concentration, and, therefore, it was possible to detect CRP with high sensitivity. In addition, since a change in the polarization anisotropy was able to be grasped even in a state in which the concentration of the affinity particle was low and was 0.0001 mg/mL, it was ascertained that the affinity particle exhibited intense light emission.

As is clear from the present example, an affinity particle that has high detection sensitivity to a living-body component and that is usable for the fluorescent depolarization method is provided.

The embodiments according to the present disclosure include the following configurations and method.

### Configuration 1

A resin particle including a europium complex,
wherein the resin particle has a core-shell structure,
a core portion of the resin particle contains a copolymer having a unit denoted by

Formula (CORE_1) below,
a shell portion of the resin particle contains a polymer having a unit denoted by Formula (SHELL_1) below and a polymer having a unit denoted by Formula (SHELL_2) below, and
at least one of the core portion and the shell portion includes a cross-linked structure:

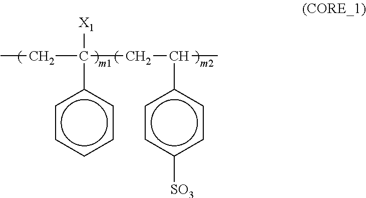

in Formula (CORE_1), X₁ represents H or CH₃, each of m1 and m2 represents an integer of 1 or more, and m1 and m2 satisfy 10<m1/m2.

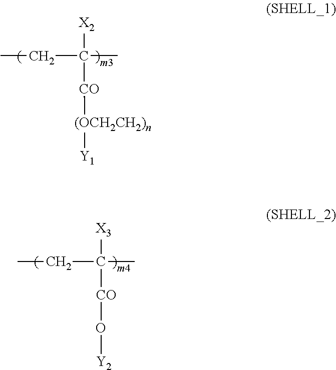

In Formula (SHELL_1) and Formula (SHELL_2), each of X₂ and X₃ represents H or CH₃, Y₁ represents OH or OCH₃, Y₂ represents Formula (SHELL_3) below or CH₂CH₂OH, each of m3 and m4 represents an integer of 1 or more, and n represents an integer of 1 or more and 40 or less.

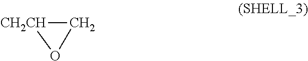

### Configuration 2

The resin particle according to Configuration 1, wherein the europium complex is denoted by Formula (COMP_1) below.

Eu(A)ₓ(B)_{y}(C)_{z} (COMP_1)

In Formula (COMP_1), (A) is a ligand denoted by Formula (COMP_2) below, (B) is a ligand denoted by Formula (COMP_3) or Formula (COMP_4) below, and (C) is a ligand denoted by Formula (COMP_5) below.

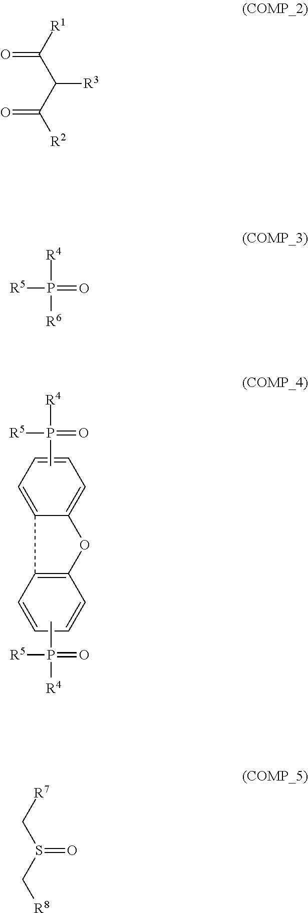

In Formula (COMP_2) to Formula (COMP_5), each of R¹ and R² represents an alkyl group, a perfluoroalkyl group, a phenyl group, or a thiophene group that may have a substituent, R³ represents a hydrogen atom or a methyl group, each of R⁴ and R⁵ represents an alkyl group or a phenyl group that may have a substituent, R⁶ represents an alkyl group, a phenyl group, or a triphenylene group that may have a substituent, each of R⁷ and R⁸ represents an alkyl group or a phenyl group that may have a substituent, in Formula (COMP_4), a bond indicated by a dotted line is not limited to being present, each substituent is any one of a methyl group, a fluoro group, a chloro group, and a bromo group, a carbon number of each alkyl group is 2 or more and 12 or less, and x, y, and z satisfy Formula (COMP_6), Formula (COMP_7), Formula (COMP_8), and Formula (COMP_9) below.

$$x = 3 ( COMP\_ 6 )$$

$$y = 1 or 2 ( COMP\_ 7 )$$

$$z = 0 or 1 ( COMP\_ 8 )$$

$$x + y + z = 4 or 5 ( COMP\_ 9 )$$

### Configuration 3

The resin particle according to Configuration 2, wherein the europium complex is denoted by Formula (COMP_10) or Formula (COMP_11) below.

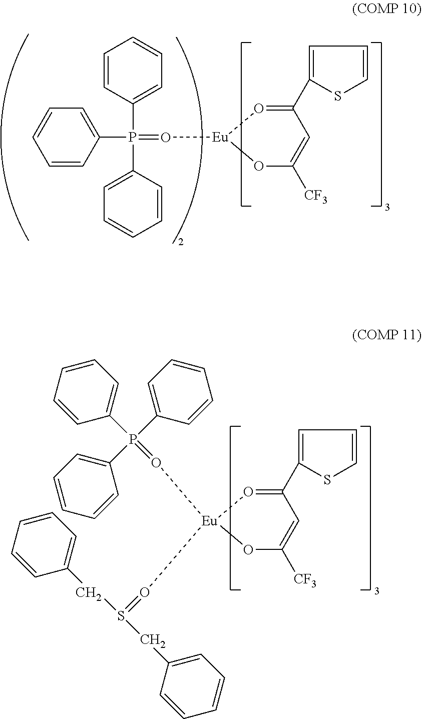

### Configuration 4

The resin particle according to any one of Configurations 1 to 3, wherein a content of the europium complex per g of the resin particle is 0.001 g or more.

### Configuration 5

The resin particle according to any one of Configurations 1 to 4, wherein the shell portion contains at least one selected from the group consisting of a polymer of hydroxyethyl methacrylate, a polymer of polyethylene glycol monomethyl ether methacrylate, a polymer of 2-methoxyethyl acrylate, a polymer of 2-methoxyethyl methacrylate, and a polymer of glycidyl methacrylate.

### Configuration 6

The resin particle according to any one of Configurations 1 to 5, wherein the cross-linked structure included in the shell portion is formed by using trimethylolpropane trimethacrylate.

### Configuration 7

The resin particle according to any one of Configurations 1 to 6, wherein the cross-linked structure included in the core portion is formed by using divinylbenzene.

### Configuration 8

The resin particle according to any one of Configurations 1 to 7, wherein a relationship between a radius (D1) of the core portion and a film thickness (D2) of the shell portion is denoted by Formula (RA_1) below.

$$50 > D 1 / D 2 > 5 / 3 ( RA\_ 1 )$$

### Configuration 9

The resin particle according to any one of Configurations 1 to 8, wherein a diameter of the resin particle is 80 nm or more and 200 nm or less.

### Configuration 10

An affinity particle including
the resin particle according to any one of Configurations 1 to 9 and
a ligand containing at least one selected from the group consisting of an antibody, an antigen, a protein, and a nucleic acid,
wherein the shell portion has at least one functional group selected from the group consisting of a carboxy group, an amino group, a thiol group, an epoxy group, a maleimide group, and a succinimidyl group, and
the ligand is bonded to the functional group.

### Configuration 11

The affinity particle according to Configuration 10 used to detect a target substance by using a fluorescent depolarization method.

### Method 1

A detection method including
obtaining a liquid mixture by mixing the affinity particle according to Configuration 10 or Configuration 11 and a specimen having possibility of containing a target substance,
applying polarized light to the liquid mixture, and
separating and detecting a polarization component in the light emission from the affinity particle in the liquid mixture.

According to the resin particle of the present disclosure, a resin particle that does not readily cause nonspecific adsorption when being used to detect a target substance such as an antigen is provided. In particular, a resin particle having high sensitivity when a target substance is detected by using a fluorescent depolarization method is provided.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2024-201551, filed Nov. 19, 2024 and Japanese Patent Application No. 2023-205719, filed Dec. 5, 2023, each of which is hereby incorporated by reference herein in their entirety.

What is claimed is:

1. A resin particle having a core-shell structure, comprising:
a europium complex;
a core portion; and
a shell portion
wherein the core portion contains a copolymer having a unit denoted by Formula (CORE_1),
the shell portion contains a polymer having a unit denoted by Formula (SHELL_1) and a polymer having a unit denoted by Formula (SHELL_2), and
at least one of the core portion and the shell portion includes a cross-linked structure: 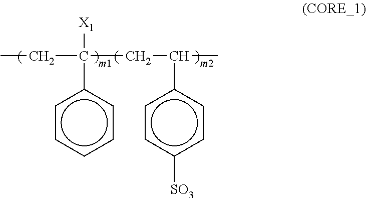
in Formula (CORE_1), X₁ represents H or CH₃, each of m1 and m2 represents an integer of 1 or more, and m1 and m2 satisfy 10<m1/m2, 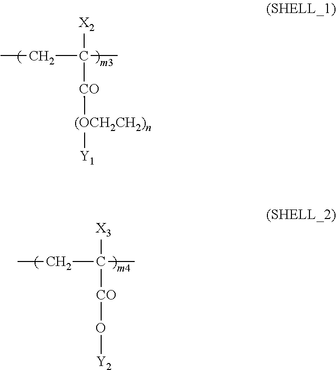
in Formula (SHELL_1) and Formula (SHELL_2), each of X₂ and X₃ represents H or CH₃, Y₁ represents OH or OCH₃, Y₂ represents Formula (SHELL_3) or CH₂CH₂OH, each of m3 and m4 represents an integer of 1 or more, and n represents an integer of 1 or more and 40 or less,

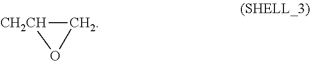

2. The resin particle according to claim 1,
wherein the europium complex is denoted by Formula (COMP_1):
Eu(A)ₓ(B)_{y}(C)_{z} (COMP_1)
in Formula (COMP_1), (A) is a ligand denoted by Formula (COMP_2), (B) is a ligand denoted by Formula (COMP_3) or Formula (COMP_4), and (C) is a ligand denoted by Formula (COMP_5), 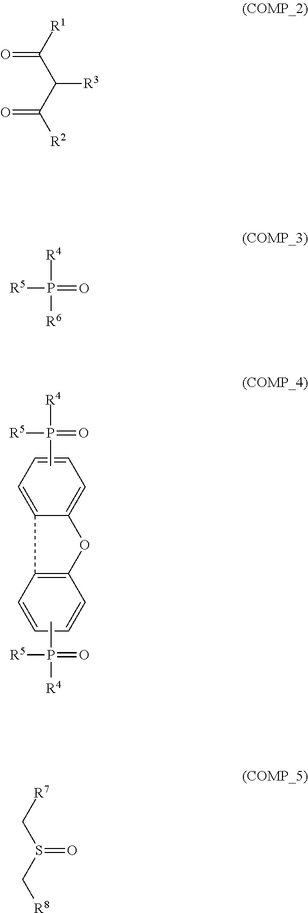
in Formula (COMP_2) to Formula (COMP_5), each of R¹ and R² represents an alkyl group, a perfluoroalkyl group, a phenyl group, or a thiophene group that may have a substituent, R³ represents a hydrogen atom or a methyl group, each of R⁴ and R⁵ represents an alkyl group or a phenyl group that may have a substituent, R⁶ represents an alkyl group, a phenyl group, or a triphenylene group that may have a substituent, each of R⁷ and R⁸ represents an alkyl group or a phenyl group that may have a substituent, in Formula (COMP_4), a bond indicated by a dotted line is not limited to being present, each substituent is any one of a methyl group, a fluoro group, a chloro group, and a bromo group, a carbon number of each alkyl group is 2 or more and 12 or less, and
x, y, and z satisfy Formula (COMP_6), Formula (COMP_7), Formula (COMP_8), and Formula (COMP_9):
$$x = 3 ( COMP\_ 6 )$$
$$y = 1 or 2 ( COMP\_ 7 )$$
$$z = 0 or 1 ( COMP\_ 8 )$$
$$x + y + z = 4 or 5 ( COMP\_ 9 ) .$$

3. The resin particle according to claim 2,
wherein the europium complex is denoted by Formula (COMP_10) or Formula

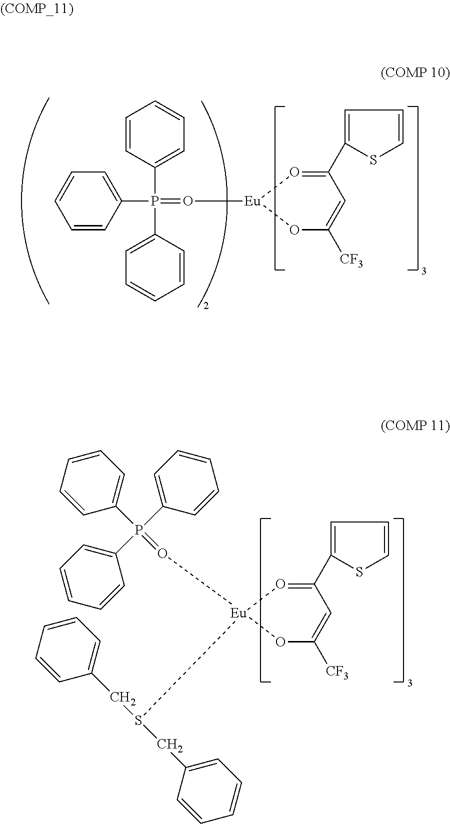

4. The resin particle according to claim 1,
wherein a content of the europium complex per g of the resin particle is 0.001 g or more.

5. The resin particle according to claim 1,
wherein the shell portion contains at least one polymer selected from the group consisting of a polymer of hydroxyethyl methacrylate, a polymer of polyethylene glycol monomethyl ether methacrylate, a polymer of 2-methoxyethyl acrylate, a polymer of 2-methoxyethyl methacrylate, and a polymer of glycidyl methacrylate.

6. The resin particle according to claim 1,
wherein the cross-linked structure included in the shell portion is formed by using trimethylolpropane trimethacrylate.

7. The resin particle according to claim 1,
wherein the cross-linked structure included in the core portion is formed by using divinylbenzene.

8. The resin particle according to claim 1,
wherein a relationship between a radius (D1) of the core portion and a film thickness (D2) of the shell portion is denoted by Formula (RA_1):
50 >D1/D2 >5/3 (RA_1).

9. The resin particle according to claim 1,
wherein a diameter of the resin particle is 80 nm or more and 200 nm or less.

10. The resin particle according to claim 1,
wherein a polydispersity index of the resin particle is 0.1 or less.

11. An affinity particle comprising:
the resin particle according to claim 1 and
a ligand containing at least one selected from the group consisting of an antibody, an antigen, a protein, and a nucleic acid,
wherein the shell portion has at least one functional group selected from the group consisting of a carboxy group, an amino group, a thiol group, an epoxy group, a maleimide group, and a succinimidyl group, and
the ligand is bonded to the functional group.

12. The affinity particle according to claim 11 used to detect a target substance by using a fluorescent depolarization method.

13. A detection method comprising:
obtaining a liquid mixture by mixing the affinity particle according to claim 12 and a specimen having possibility of containing a target substance;
applying polarized light to the liquid mixture; and
separating and detecting a polarization component in the light emission from the affinity particle in the liquid mixture.
